(19) United States
(12) Patent Application Publication
(10) Pub. No.: US 2025/0153399 A1
(43) Pub. Date: May 15, 2025

(54) ON-THE-FLY MOLDING

(71) Applicant: 3DSIL LTD., Rosh Haayin (IL)

(72) Inventors: Yair Ramot, Maas (IL); Roi Ramot, Rosh Haayin (IL)

(73) Assignee: 3DSIL LTD., Rosh Haayin (IL)

(21) Appl. No.: 19/024,197

(22) Filed: Jan. 16, 2025

Related U.S. Application Data
(63) Continuation of application No. 17/796,733, filed on Aug. 1, 2022.
(60) Provisional application No. 62/969,107, filed on Feb. 2, 2020.

(51) Int. Cl. B29C 33/38; A61F 2/00; A61F 2/12; B29C 64/118; B29C 64/209; B33Y 10/00; B33Y 30/00; B33Y 80/00

(52) U.S. Cl. CPC B29C 33/3842; A61F 2/0059; A61F 2/12; B29C 64/118; B29C 64/209; A61F 2240/002; A61F 2240/004; B33Y 10/00; B33Y 30/00; B33Y 80/00

(57) ABSTRACT

A method of fabricating an object from a heat curable polymer such as silicone is provided. The method is carried out by using additive manufacturing to fabricate a portion of a mold and filling the portion of the mold with the heat curable polymer. The polymer is then heated and the steps of mold fabrication and filling are repeated until the object is fabricated.

### FIELD OF THE INVENTION

The present invention relates to a method of fabricating heat-curable polymer objects, such as implants.

### BACKGROUND

Medical implants that are customized to a specific patient and address a clinical need have become a reality in recent years, and a customized implant is a vastly superior clinical solution for many patients.

Silicone rubber is highly suitable for use in medical implants due to a lack of reactivity with the human body and the fact that it is easily molded into any desired shape and holds its shape for extended periods of time.

Silicone implants are made from medical grade silicone (FDA/CE approved, e.g., NUSIL) with varying properties (e.g., Shore hardness levels) and are typically manufactured using high volume methods, such as injection molding, compression molding or rotary molding. These approaches require the design and manufacturing of a dedicated mold and the certification of the molding process for every product. This is a lengthy and costly process, and as such, it is not well suited for customized implants.

Customized silicone implants are typically fabricated by manually carving silicone blocks; fabrication is typically carried out by the surgeon using a knife. This approach is relatively inexpensive but highly inaccurate and surgeon-specific and as such, surgical outcomes of surgeries that utilize carved block implants vary in quality.

There is thus a need for, and it would be highly advantageous to have, an inexpensive and relatively fast approach for fabricating highly accurate and personalized silicone implants.

### SUMMARY

According to one aspect of the present invention there is provided a method of fabricating an object from a heat curable polymer including (a) using additive manufacturing to fabricate a portion of a mold; (b) filling the portion of the mold with the heat curable polymer; (c) heating the polymer; and repeating steps (a)-(c) to fabricating the object.

According to embodiments of the present invention steps (a) and (b) are performed simultaneously.

According to embodiments of the present invention the heat curable polymer is a medical grade silicone.

According to embodiments of the present invention the object is a medical implant.

According to embodiments of the present invention steps (b) and (c) are performed simultaneously.

According to embodiments of the present invention step (c) is performed using a heated polymer-delivery nozzle.

According to embodiments of the present invention step (a) is performed using 3D printing.

According to embodiments of the present invention the mold is manufactured from a dissolvable material.

According to embodiments of the present invention the dissolvable material can be High Impact Polystyrene (HIPS) or Polyvinyl Alcohol (PVA) or any other dissolvable element.

According to embodiments of the present invention (a) and (b) are performed using side-by-side print nozzles.

According to another aspect of the present invention there is provided a system for fabricating an object from a heat curable polymer including: (a) a first nozzle configured for additive manufacturing of a mold; (b) a second nozzle for filling the mold with a heat curable polymer; and (c) a heating element for heating the polymer in the mold.

According to embodiments of the present invention the first and the second nozzles are side-by-side print nozzles.

According to embodiments of the present invention the system further includes a first reservoir for storing a dissolvable material for additive manufacturing of the mold.

According to embodiments of the present invention the system further includes at least one additional reservoir for storing liquid components of the heat curable polymer.

According to embodiments of the present invention the heat curable polymer is medical grade silicone.

According to embodiments of the present invention the heating element is positioned in proximity to, or in contact with, the second nozzle.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings:

FIG. 1 is a flowchart outlining the fabrication process according to one embodiment of the present invention.

FIGS. 2A-B schematically illustrate a configuration of the present system which includes a dual nozzle printing head (FIG. 2A) and two independent printing heads (FIG. 2B).

FIG. 3 illustrates a silicone implant fabricated within a perishable mold.

### DETAILED DESCRIPTION

The present invention provides a method which can be used to fabricate a customized implant from a medical grade heat-curable polymer. Specifically, the present invention can be used to fabricate a customized implant from implantable (and certified) medical grade silicone.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Silicone elastomer is formed by crosslinking silicone polymer chains via an addition reaction between the vinyl functional groups of a vinyl silicone polymer and the silicon hydride of a crosslinking agent containing SiH functions. The reaction requires the presence of a catalyst, usually an organometallic complex of platinum. Medical-grade silicone implants are fabricated from certified silicone component (raw materials) and a certified manufacturing process by heat curing the mixed components at the manufacturer's specified parameters of temperature and curing time.

In order to meet FDA/CE regulations the implant material must fully pass verification and validation testing including a bio-compatibility test and clinical testing as defined by the regulatory authorities (FDA/CE).

Raw silicone cannot be readily used in additive manufacturing approaches since the relatively lengthy cure time renders it unsuitable for rapid manufacturing.

Additive manufacturing of silicone components utilizes silicones that are chemically modified to enable rapid polymerization suitable for 3D printing.

Any modification of raw silicone renders a product manufactured thereby unsuitable for use as an implant since addition of components to the raw material redefines the silicone product as a new material that has to be recertified by regulatory authorities (including certification of the specific manufacturing process).

Thus, while modified silicones can be 3D printed, the resultant product does not meet regulatory guidelines and as such it cannot be used for fabrication of medical implants.

While reducing the present invention to practice, the present inventors have devised an approach for fabricating implants from a heat-curable medically approved for implant devices polymer such as silicone.

Thus, according to one aspect of the present invention there is provided a method of fabricating an object from a heat curable polymer.

The heat-curable polymer can be any single or multi-component polymer and is preferably approved for medical use or for use in the food and health industries. Examples of such polymers include silicone (NUSIL 48XX), polychloroprene (CR)/neoprene, ethylene propylene diene monomer (EPDM), fluoroelastomers (FKM)/Viton and acrylic rubber (ACM).

The object can be, for example, a medical implant used in orthopedic or reconstructive/aesthetic/corrective surgery. Such an implant can be, for example, breast implants, pectoral implants, facial and ear reconstruction implants, stents, indwelling catheters and the like.

The method of the present invention is carried by fabricating a portion of a mold using additive manufacturing and filling that portion with the heat curable polymer. The polymer is then heated to a solid or semi-solid state and the step of mold fabrication and polymer filling and curing is repeated one or more times until the object is completely fabricated. The object can then be freed from the mold by, for example, dissolving the mold material.

Such an approach, which is termed herein as “on-the-fly” molding enables:
(i) rapid and accurate fabrication of a variety of custom-made medical implants;
(ii) use of medical grade heat curable polymers such as silicone;
(iii) creation of structures having thin elements and texturing; and
(iv) creation of complex designs with nearly completely enclosed cavities.

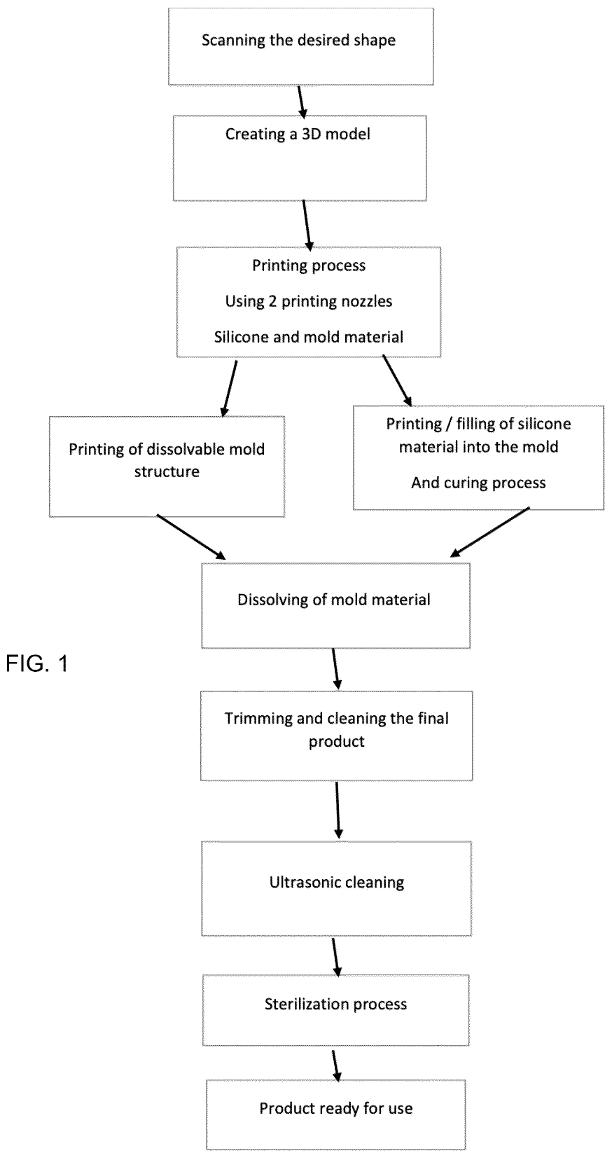

Referring now to the drawings, FIG. 1 is a flowchart outlining the steps of fabricating a medical implant via the present approach.

In order to fabricate a customized implant, the desired shape of the implant is first determined and modeled. For example, in cases where the implant augments or replaces an existing anatomical structure an imaging modality (e.g. X-ray, MRI) is first used to scan the anatomical structure and generate a 3D model using well known approaches.

The model can then be used to generate a model of the structure and needed mold using a CAD/CAM program and/or a specially designed and customized software for creating the printing file directly from pictures or any other scanning technology

The customized software computes and sets all the needed printing parameters, such as printing increments, sequence and mold design.

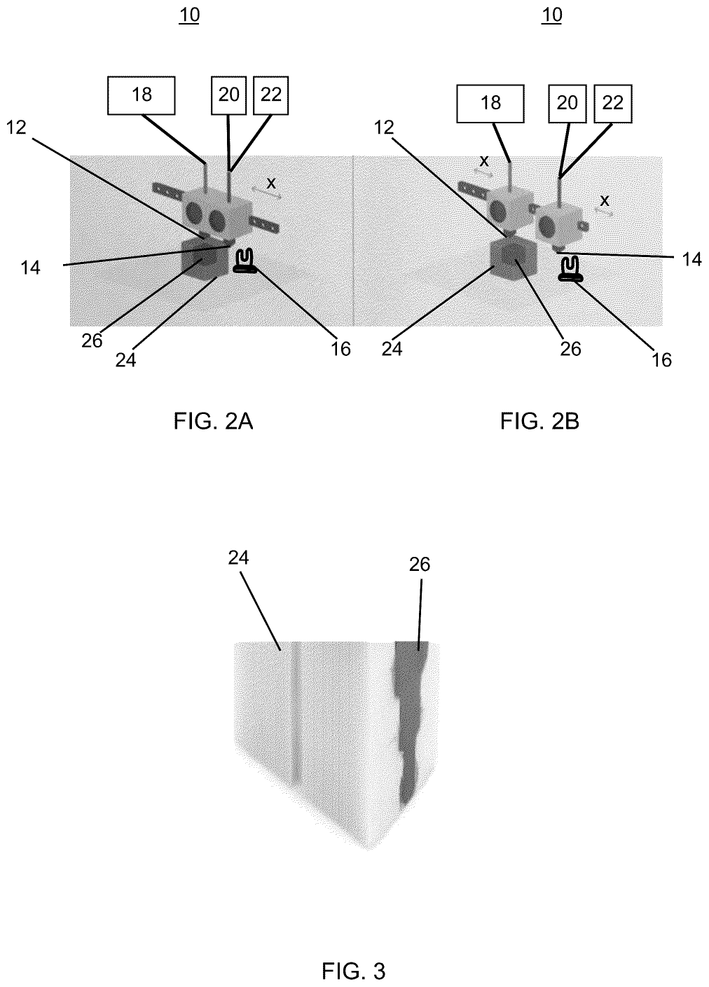

The mold parameters are then fed into a dual/two nozzle system capable of 3D printing the mold and injecting the heat-curable polymer (silicone) into the mold. Such a system is further described hereinbelow with reference to FIGS. 2A-B.

The system prints the mold and fills it in a stepwise or continuous fashion. The resolution of each step of the printing process can be determined by the structure of the printed part and the precision needed for that product. For simple objects that are largely volumetric (e.g., simple aesthetic implants) the process can be simultaneous with the mold being filled with the silicone as its being fabricated. For objects of more complex shapes, a stepwise mold building and filling process can be used. The mold can be fabricated with channels that facilitate mold filling with the polymer.

As is mentioned hereinabove, the polymer used by the present invention is heat curable. Curing can be effected during mold filling and/or following mold filling. In any case, curing can be effected using a heated nozzle or a heated environment. Curing can be partial during fabrication and completed in an oven following completion of the object. Curing is effected using the polymer recommended heat and time.

Once the implant is completely fabricated and cured it can extracted from the mold (e.g., pulled out) or the mold can be dissolved (in the case of HIPS or PVA mold material). The final implant can then be trimmed to remove residual polymer elements created by the mold structure or imperfections and cleaned, ultrasonically treated (in detergent) and sterilized (gamma or autoclave) and packaged for use.

Since the present approach enables rapid and accurate fabrication of a medical implant it can be used in the hospital setting to fabricate an implant prior to or during surgery. An added advantage of the present approach is in the ability to produce several variations of an implant and to test each for fit within the timeframe of surgery.

FIGS. 2A-2B illustrate two configurations of a system 10 that can be used to carry out the fabrication process of the present invention.

System 10 can include a 3D printer having a 3D (X, Y, Z) stage, two printing heads each fitted with a nozzle. The printing head and nozzle for printing the mold can be a standard 3D printing head. The printing head and nozzle for dispensing the polymer can be configured for mixing the two components of the polymer (at the correct mixing ratio) and dispensing the mixed material though the printing nozzle. The apparatus may have a specially designed heating system including a heat extracting nozzle that can cure the printed polymer. System 10 can include an enclosure for creating an airless atmosphere in close proximity to the fabricated object to prevent unwanted air cavities (bubbles) inside the printed mold or injected object.

System 10 is configured for additive manufacturing using a first nozzle 12 and for injection of a heat curable polymer using nozzle 14 and heating element 16.

System 10 of FIG. 2A includes a single movable head with nozzles 12 and 14 mounted thereupon. System 10 of FIG. 2B includes two heads, each fitted with a nozzle. Thus, in the configuration of FIG. 2A, nozzles 12 and 14 move together in the X-axis (arrow) while in the configuration of FIG. 2B nozzles 12 and 14 are independently movable (along the X-axis, arrows).

Nozzle 12 is for printing the dissolvable mold material using FDM (fused deposition modeling) technology including a conveying disposing material system that pushes a wire material through the nozzle. Nozzle 14 is for printing the polymer material. It consists of a nozzle, a conveying disposing system that presses the mixed polymer material through the specially designed nozzle and setting the correct volume for the fabricated object. It also includes an automatic mixing system for mixing the two components of the polymer.

System 10 further includes reservoir 18 or material wire cassette for feeding the mold material to nozzle 12 and reservoirs 20 and 22 for feeding the liquid components of the heat-curable polymer to nozzle 14. In the case of silicone, the components are independently fed from reservoirs 20 and 22 and are mixed (e.g., using a mixer) prior to being pushed into nozzle 14. Once injected into a mold 24, a heat-curable polymer 26 is completely or partially cured using heating element 16.

It will be appreciated that a single reservoir can also be used for the heat-curable polymer. Such a reservoir can be filled with the premixed liquid. Heat element 16 can be integrated into nozzle 14 or positioned in close proximity (e.g., 1-10 mm) to nozzle 14 or from mold 24. As is described hereinabove, polymer 26 is injected into mold 24 during mold production (stepwise or simultaneous). In order to enable such functionality, system 10 is programmed to first print the mold from dissolvable material using nozzle 12. Once a first volumetric layer of the mold is fabricated, the polymer is printed/filled into that layer. These steps are repeated for each layer. A software controlling fabrication can automatically set the proper sequence of fabrication and can determine the printing increments based on accuracy and speed specified by final product specifications or user.

FIG. 3 illustrates mold 24 and fabricated implant 26 prior to implant extraction.

It is expected that during the life of this patent many relevant heat-curable medical grade polymers will be developed and the scope of the term heat-curable polymer is intended to include all such new technologies a priori.

As used herein the term “about” refers to ±10%.

What is claimed is:

1. A method of fabricating an object comprising:
(a) using additive manufacturing to fabricate a portion of a mold;
(b) filling said portion of said mold with a heat curable polymer that passes verification, validation, bio-compatibility, and clinical testing defined by the Food and Drug Administration such that said heat curable polymer is called a medical grade heat curable polymer;
(c) heating said heat curable polymer;
(d) repeating steps (a)-(c) to fabricating the object, wherein said heat curable polymer is still said medical grade heat curable polymer after fabricating the object.

2. The method of claim 1, wherein (a) and (b) are performed simultaneously.

3. The method of claim 1, wherein the heat curable polymer is a medical grade silicone.

4. The method of claim 1, wherein the object is a medical implant.

5. The method of claim 1, wherein (b) and (c) are performed simultaneously.

6. The method of claim 1, wherein (c) is performed using a heated polymer-delivery nozzle.

7. The method of claim 1, wherein (a) is performed using 3D printing.

8. The method of claim 1, wherein said mold is manufactured from a dissolvable material.

9. The method of claim 8, wherein said dissolvable material can be High Impact Polystyrene (HIPS) or Polyvinyl Alcohol (PVA) or any other dissolvable element.

10. The method of claim 1, wherein (a) and (b) are performed using side-by-side print nozzles.
